(19) United States
(12) Patent Application Publication
(10) Pub. No.: US 2021/0269602 A1
(43) Pub. Date: Sep. 2, 2021

(54) METHOD FOR PRODUCING STEREOCOMPLEX POLYLACTIC ACID COMPOSITE BASED ON OIL-IN-WATER EMULSION BLENDING, METHOD FOR PREPARING DRUG DELIVERY COMPOSITION USING STEREOCOMPLEX POLYLACTIC ACID COMPOSITE PRODUCED BY THE PRODUCTION METHOD AND DRUG DELIVERY COMPOSITION PREPARED BY THE PREPARATION METHOD

(71) Applicant: KOREA INSTITUTE OF SCIENCE AND TECHNOLOGY, Seoul (KR)

(72) Inventors: Soo Hyun KIM, Seoul (KR); Youngmee JUNG, Seoul (KR); Justin Jihong CHUNG, Seoul (KR); Seung Hyuk IM, Seoul (KR)

(73) Assignee: KOREA INSTITUTE OF SCIENCE AND TECHNOLOGY, Seoul (KR)

(21) Appl. No.: 17/095,925

(22) Filed: Nov. 12, 2020

(30) Foreign Application Priority Data
Feb. 28, 2020 (KR) 10-2020-0025077

(51) Int. Cl. C08J 3/03; A61K 9/107; A61K 47/34

(52) U.S. Cl. CPC C08J 3/03; C08J 2367/04; A61K 47/34; A61K 9/107

(57) ABSTRACT

Provided are a method for producing a stereocomplex polylactic acid composite based on oil-in-water emulsion blending, a method for preparing a drug delivery composition using a stereocomplex polylactic acid composite produced by the production method, and a drug delivery composition prepared by the preparation method. The stereocomplex polylactic acid composite is produced using oil-in-water emulsion blending that has the advantages of simple process, short production time, and high stereocomplexation efficiency compared to existing methods such as solution blending, melt-blending, and supercritical fluid technology. In addition, the drug delivery composition is prepared using oil-in-water emulsion blending that allows the drug to be loaded into the polymer chains of the stereocomplex polylactic acid in a very easy and efficient manner and facilitates release of the drug at room temperature over a long period of time.

### CROSS-REFERENCE TO RELATED APPLICATIONS

This application claims priority under 35 U.S.C. § 119 to Korean Patent Application No. 10-2020-0025077 filed on Feb. 28, 2020 in the Korean Intellectual Property Office, the disclosure of which is incorporated herein by reference in its entirety.

### BACKGROUND OF THE INVENTION

### 1. Field of the Invention

The present invention relates to a method for producing a stereocomplex polylactic acid composite based on oil-in-water emulsion blending, a method for preparing a drug delivery composition using a stereocomplex polylactic acid composite produced by the production method, and a drug delivery composition prepared by the preparation method.

### 2. Description of the Related Art

Polylactic acid (PLA) or polylactide is a promising biodegradable polymer and exists as two different enantiomers, i.e. L- and D-forms, in nature. The two L- and D-form enantiomers combine to form a stereocomplex (sc) crystal. The resulting stereocomplex polylactic acid (sc-PLA) has greatly improved physical and mechanical strength as well as excellent thermal properties compared to the homopolymers. Based on these characteristics, environmentally friendly polymers based on the stereocomplex polylactic acid can be utilized as implantable materials and components that should withstand high external forces and pressures.

A stereocomplex polylactic acid is usually produced by solution blending, melt-blending or supercritical fluid technology. However, these conventional methods have many problems such as poor processability, low yield, low solubility, and high production cost. For example, solution blending is the easiest and widely used method because the process is very simple and readily accessible but has the disadvantages of very low stereocomplexation efficiency and long reaction time.

Melt-blending has the advantages of high stereocomplexation efficiency and short reaction time compared to solution blending but is disadvantageous in that since it induces the formation of a stereocomplex polylactic acid composite in a state in which the polymer is melted by heating, the molecular weight of the polymer is reduced and the original properties of the polymer are impaired. Supercritical fluid technology has the advantages of higher stereocomplexation efficiency, higher solubility, and much shorter reaction time than the other two methods. However, supercritical fluid technology involves an extremely complex process and uses high pressure, posing an increased danger.

### PRIOR ART DOCUMENTS

### Patent Documents

(Patent Document 1) Korean Patent No. 10-1386399

### SUMMARY OF THE INVENTION

The present invention has been made in an effort to solve the above problems, and one object of the present invention is to provide a method for producing a stereocomplex polylactic acid composite based on oil-in-water emulsion blending that has the advantages of short synthesis time and high stereocomplexation efficiency compared to solution blending, melt-blending or supercritical fluid technology.

A further object of the present invention is to provide a stereocomplex polylactic acid composite with high thermal stability and crystallinity.

Another object of the present invention is to provide a method for preparing a drug delivery composition using the stereocomplex polylactic acid composite.

Another object of the present invention is to provide a drug delivery composition that readily releases a drug at room temperature over a long period of time.

Objects of the present invention are not limited to the above-mentioned ones. The objects of the present invention will become more apparent from the following detailed description and will be implemented by means described in the claims and a combination thereof.

The present invention provides a method for producing a stereocomplex polylactic acid composite based on oil-in-water emulsion blending, including: (a) preparing a water phase solution including water and a first nonionic surfactant; (b) preparing an oil phase solution including poly(L-lactic acid) and poly(D-lactic acid); (c) dispersing the oil phase solution in the water phase solution while adding dropwise the oil phase solution at a rate of 1 to 200 μl/s to the water phase solution, to prepare an oil-in-water (O/W) emulsion; and (d) stirring the oil-in-water emulsion.

The water phase solution may be prepared by mixing the water with the first nonionic surfactant in a weight ratio of 1:0.01-0.5.

In step (b), the oil phase solution may be prepared by mixing the poly(L-lactic acid) with the poly(D-lactic acid) in a weight ratio of 1:9 to 9:1 for 1 minute to 3 hours or for 15 hours to 28 hours.

The oil phase solution may further include 0.1 to 10% by weight of a second nonionic surfactant, based on the total weight thereof.

The first nonionic surfactant and the second nonionic surfactant may be each independently selected from the group consisting of polyoxyethylene sorbitan monolaurate, polyoxyethylene sorbitan monostearate, polyoxyethylene sorbitan monooleate, polyoxyethylene lauryl ether, polyoxyethylene cetyl ether, polyoxyethylene oleyl ether, polyoxyethylene, and mixtures thereof.

In step (c), the oil-in-water (O/W) emulsion may be prepared by adding dropwise the oil phase solution at a rate of 15 to 80 μl/s to the water phase solution with a micropipette.

In step (d), the oil-in-water emulsion may be subjected to magnetic stirring at a speed of 100 to 1000 rpm.

The stereocomplex polylactic acid composite may be in the form of microspheres having an average particle diameter of 0.1 to 100 μm and may have a melting point of 200 to 280° C. and a stereocomplexation efficiency of at least 95%.

The water phase solution may be prepared by mixing the water with the first nonionic surfactant in a weight ratio of 1:0.03-0.08; the oil phase solution may be prepared by mixing the poly(L-lactic acid) with the poly(D-lactic acid) in a weight ratio of 5:5 for 1 minute to 5 minutes in step (b); the oil phase solution may further include 1 to 6% by weight of a second nonionic surfactant, based on the total weight thereof; each of the first nonionic surfactant and the second nonionic surfactant may be polyoxyethylene sorbitan monolaurate; and the oil-in-water (O/W) emulsion may be prepared by adding dropwise the oil phase solution at a rate of 20 to 50 μl/s to the water phase solution with a micropipette in step (c).

The present invention provides a stereocomplex polylactic acid composite produced by mixing an oil phase solution including poly(L-lactic acid) and poly(D-lactic acid) with a water phase solution including water and a nonionic surfactant and subjecting the mixture to oil-in-water emulsion blending wherein the stereocomplex polylactic acid composite is in the form of microspheres having an average particle diameter of 0.1 to 100 μm and has a melting point of 200 to 280° C. and a stereocomplexation efficiency of at least 95%.

The poly(L-lactic acid) may have a number average molecular weight (Mn) of 50,000 to 150,000 g/mol and the poly(D-lactic acid) may have a number average molecular weight (Mn) of 50,000 to 150,000 g/mol.

The stereocomplex polylactic acid composite may be a mixture of the poly(L-lactic acid) and the poly(D-lactic acid) in a weight ratio of 1:9 to 9:1.

The present invention also provides a method for preparing a drug delivery composition, including: (a) preparing a water phase solution including water and a first nonionic surfactant; (b) preparing an oil phase solution including poly(L-lactic acid), poly(D-lactic acid), and a drug; (c) dispersing the oil phase solution in the water phase solution while adding dropwise the oil phase solution at a rate of 1 to 200 μl/s to the water phase solution to prepare an oil-in-water (O/W) emulsion; and (d) stirring the oil-in-water emulsion to produce a stereocomplex polylactic acid composite loaded with the drug.

In step (b), the oil phase solution may be prepared by adding 0.1 to 33% by weight of the drug with respect to the total weight thereof, followed by mixing for 1 minute to 3 hours or for 15 hours to 18 hours.

The drug may be selected from the group consisting of fluorouracil (5-FU), capecitabine, cytarabine, gemcitabine, mercaptopurine, fludarabine, methotrexate, pemetrexed, and mixtures thereof.

The present invention also provides a drug delivery composition prepared by mixing an oil phase solution including poly(L-lactic acid), poly(D-lactic acid), and a drug with a water phase solution including water and a nonionic surfactant and subjecting the mixture to oil-in-water emulsion blending to produce a stereocomplex polylactic acid composite loaded with the drug wherein the stereocomplex polylactic acid composite is in the form of microspheres having an average particle diameter of 0.1 to 100 μm and has a melting point of 200 to 280° C. and a stereocomplexation efficiency of at least 95%.

The drug may be present in an amount of 0.1 to 33% by weight, based on the total weight of the drug delivery composition.

The drug may be selected from the group consisting of fluorouracil (5-FU), capecitabine, cytarabine, gemcitabine, mercaptopurine, fludarabine, methotrexate, pemetrexed, and mixtures thereof.

The stereocomplex polylactic acid composite of the present invention is produced using oil-in-water emulsion blending that has the advantages of simple process, short production time, and high stereocomplexation efficiency compared to existing methods such as solution blending, melt-blending, and supercritical fluid technology.

In addition, the drug delivery composition using the stereocomplex polylactic acid composite of the present invention is prepared using oil-in-water emulsion blending that allows the drug to be loaded into the polymer chains of the stereocomplex polylactic acid in a very easy and efficient manner and facilitates release of the drug at room temperature over a long period of time.

Effects of the present invention are not limited to the above-mentioned ones. It is should be understood that the effects of the present invention include all effects inferable from the following detailed description.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIG. 1 is a diagram schematically showing a method for producing a stereocomplex polylactic acid composite based on oil-in-water emulsion blending according to the present invention;

FIG. 2A graphically shows the results of thermal analysis, FIG. 2B shows thermogravimetric analysis, and FIG. 2C shows wide-angle X-ray diffraction analysis for an scPLA composite produced in Example 1 (Emulsion 5), PLLA, and PDLA;

FIG. 3 shows the particle sizes of sc-PLA composites produced in Example 1 (Emulsions 1 to 5);

FIGS. 4A and 4D show the stereocomplexation efficiencies of stereocomplex polylactic acid composites produced in Example 1 and Comparative Examples 1-3 and FIGS. 4B and 4C show the results of thermogravimetric analysis for the stereocomplex polylactic acid composites;

FIG. 5A shows ¹H-NMR spectra, FIG. 5B shows transmittances, and FIG. 5C shows DSC thermograms of an sc-PLA composite produced in Example 1 (Emulsion 5) and a drug delivery composition (sc-PLA/5-FU) prepared in Example 2; and

FIG. 6 shows the drug release profile of an anticancer agent (5-FU) from a drug delivery composition (sc-PLA/5-FU) prepared in Example 2 for, which was determined by HPLC.

### DETAILED DESCRIPTION OF THE INVENTION

The present invention will now be described in more detail as one embodiment.

The present invention is directed to a method for producing a stereocomplex polylactic acid composite based on oil-in-water emulsion blending, a method for preparing a drug delivery composition using a stereocomplex polylactic acid composite produced by the production method, and a drug delivery composition prepared by the preparation method.

The present inventors have conducted research to overcome the limitations of conventional methods for preparing stereocomplex polylactic acid composites, and as a result, found that a stereocomplex polylactic acid composite can be produced using oil-in-water (O/W) emulsion blending while avoiding the limitations (including low stereocomplexation efficiency, low reaction speed, and complex processability) encountered in widely used methods such as solution blending, melt-blending, and supercritical fluid technology.

Specifically, the present invention provides a method for producing a stereocomplex polylactic acid composite based on oil-in-water emulsion blending, including: (a) preparing a water phase solution including water and a first nonionic surfactant; (b) preparing an oil phase solution including poly(L-lactic acid) and poly(D-lactic acid); (c) dispersing the oil phase solution in the water phase solution while adding dropwise the oil phase solution at a rate of 1 to 200 μl/s to the water phase solution, to prepare an oil-in-water (O/W) emulsion; and (d) stirring the oil-in-water emulsion.

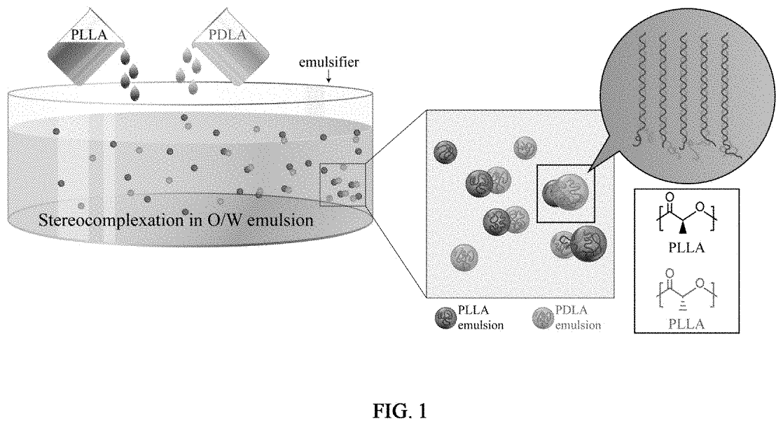

FIG. 1 is a diagram schematically showing the method for producing a stereocomplex polylactic acid composite based on oil-in-water emulsion blending according to the present invention. Referring to FIG. 1, when an oil phase solution including oil phase poly(L-lactic acid) and poly(D-lactic acid) is dispersed in a water phase solution including water and a first nonionic surfactant, the first nonionic surfactant forms an oil-in-water (O/W) emulsion consisting of two types of emulsions. The oil-in-water emulsions physically induce the production of a stereocomplex polylactic acid (Sc-PLA) composite in a delicate and rapid manner via ring-opening polymerization at the water/oil phase interface where the polymer chains of the poly(L-lactic acid) and the poly(D-lactic acid) come into contact with each other.

The individual steps of the method according to the present invention will be described in detail.

Step (a)

In step (a), a water phase solution including water and a first nonionic surfactant is prepared. The first nonionic surfactant acts as an emulsifier that disperses the oil phase solution in the water phase solution to prepare an oil-in-water emulsion in the form of microspheres. The first nonionic surfactant is preferably one having a hydrophilic lipophilic balance (HLB) of 8 to 18. Specifically, the first nonionic surfactant can be selected from the group consisting of polyoxyethylene sorbitan monolaurate, polyoxyethylene sorbitan monostearate, polyoxyethylene sorbitan monooleate, polyoxyethylene lauryl ether, polyoxyethylene cetyl ether, polyoxyethylene oleyl ether, polyoxyethylene isooctylphenyl ether, and mixtures thereof. The first nonionic surfactant is preferably selected from the group consisting of polyoxyethylene sorbitan monolaurate, polyoxyethylene sorbitan monostearate, polyoxyethylene sorbitan monooleate, and mixtures thereof. Polyoxyethylene sorbitan monolaurate is most preferred as the first nonionic surfactant.

The water phase solution may be prepared by mixing the water with the first nonionic surfactant in a weight ratio of 1:0.01-0.5, preferably 1:0.02-0.1, more preferably 1:0.03-0.08, most preferably 1:0.05. If the weight ratio of the water to the first nonionic surfactant is 1:<0.01, the oil phase solution may not be uniformly dispersed, failing to prepare an oil-in-water emulsion in the form of microspheres. Meanwhile, if the weight ratio of the water to the first nonionic surfactant is 1:>0.5, the effect of the first nonionic surfactant to further disperse the oil phase solution cannot be expected.

Step (b)

In step (b), an oil phase solution including poly(L-lactic acid) and poly(D-lactic acid) is prepared. The poly(L-lactic acid) (PLLA) and the poly(D-lactic acid) (PDLA) are biodegradable oil phase polymers. Each of these components may be dissolved in an organic solvent such as methylene chloride before use.

The poly(L-lactic acid) has a number average molecular weight (Mn) of 50,000 to 150,000 g/mol, preferably 70,000 to 135,000 g/mol, more preferably 100,000 to 120,000 g/mol, most preferably 110,000 g/mol.

The poly(D-lactic acid) has a number average molecular weight (Mn) of 50,000 to 150,000 g/mol, preferably 105,000 to 140,000 g/mol, more preferably 110,000 to 130,000 g/mol, most preferably 120,000 g/mol. If the number average molecular weight of the poly(L-lactic acid) or the poly(D-lactic acid) is outside the range of 50,000 to 150,000 g/mol, high mechanical strength, melting point, and crystallinity of the stereocomplex polylactic acid composite cannot be expected.

In step (b), the oil phase solution may be prepared by mixing the poly(L-lactic acid) with the poly(D-lactic acid) in a weight ratio of 1:9 to 9:1 for 1 minute to 3 hours or for 15 hours to 28 hours. The weight ratio of the poly(L-lactic acid) to the poly(D-lactic acid) is 1:9 to 9:1, preferably 4:6 to 6:4, most preferably 5:5. Particularly, if the mixing weight ratio is not 5:5, the thermal stability or crystallinity of the stereocomplex polylactic acid composite may deteriorate significantly.

In step (b), the mixing time is limited to 1 minute to 20 minutes, preferably 1 minute to 10 minutes, most preferably 3 minutes to 5 minutes, to prevent the oil phase solution itself from inducing the production of the stereocomplex polylactic acid composite. This limitation allows the stereocomplex polylactic acid composite to have a very small average particle diameter of 10 μm or less, reduces the time it takes to produce the composite, and improves the crystallinity of the composite.

Alternatively, the mixing time may be in the range of 20 to 28 hours, preferably 22 to 26 hours, most preferably 23 to 25 hours. Within this range, the oil phase solution induces the production of the stereocomplex polylactic acid composite. In this case, the stereocomplex polylactic acid composite has an average particle diameter of 100 μm or less.

In step (b), the mixing time is preferably limited to 1 minute to 20 minutes to prevent the oil phase solution itself from inducing the production of the stereocomplex polylactic acid composite.

The oil phase solution may further include a second nonionic surfactant to further improve the dispersibility of the poly(L-lactic acid) and the poly(D-lactic acid). The second nonionic surfactant is used in an amount of 0.1 to 10% by weight, preferably 0.5 to 8% by weight, more preferably 1 to 6% by weight, most preferably 3% by weight, based on the total weight of the oil phase solution. If the amount of the second nonionic surfactant is less than 0.1% by weight, the effect of the second nonionic surfactant to further improve the dispersibility of the oil phase solution cannot be expected. Meanwhile, if the amount of the second nonionic surfactant exceeds 10% by weight, unreacted second nonionic surfactant may remain to hinder the production of the stereocomplex polylactic acid composite.

The second nonionic surfactant may be the same as the first nonionic surfactant. Specifically, the second nonionic surfactant can be selected from the group consisting of polyoxyethylene sorbitan monolaurate, polyoxyethylene sorbitan monostearate, polyoxyethylene sorbitan monooleate, polyoxyethylene lauryl ether, polyoxyethylene cetyl ether, polyoxyethylene oleyl ether, polyoxyethylene isooctylphenyl ether, and mixtures thereof. The second nonionic surfactant is preferably selected from the group consisting of polyoxyethylene sorbitan monolaurate, polyoxyethylene sorbitan monostearate, polyoxyethylene sorbitan monooleate, and mixtures thereof. Polyoxyethylene sorbitan monolaurate is most preferred as the second nonionic surfactant.

Step (c)

In step (c), the oil phase solution is dispersed in the water phase solution while adding dropwise the oil phase solution at a rate of 1 to 200 μl/s, preferably 10 to 150 μl/s to the water phase solution to prepare an oil-in-water (O/W) emulsion. The oil-in-water emulsion may consist of two types of emulsions. Preferably, the oil-in-water emulsion is a mixture of a poly(L-lactic acid) emulsion and a poly(D-lactic acid) emulsion. Rapid dropwise addition of the oil phase solution at a high rate of 1 to 200 μl/s to the water phase solution ensures homogeneity of the poly(L-lactic acid) emulsion and the poly(D-lactic acid) emulsion to induce the production of the stereocomplex polylactic acid composite having a uniform size.

The oil-in-water emulsion is prepared by adding dropwise the oil phase solution at a high rate, more preferably a rate of 15 to 80 μl/s, most preferably a rate of 20 to 50 μl/s, to the water phase solution with a micropipette. This dropwise addition makes the polymer chains of the stereocomplex polylactic acid composite smaller such that the stereocomplex polylactic acid composite has a small average particle diameter of 3 to 6 μm. A smaller and finer average particle diameter of the stereocomplex polylactic acid composite leads to higher crystallinity, thermal stability, and mechanical stiffness of the stereocomplex polylactic acid composite.

Step (d)

In step (d), the oil-in-water emulsion is stirred using a magnetic stirrer at 100 to 1000 rpm, preferably 600 to 900 rpm, most preferably 800 rpm. If the stirring speed is lower than 100 rpm, the poly(L-lactic acid) emulsion does not readily come into contact with the poly(D-lactic acid) emulsion, increasing the time it takes to produce the stereocomplex polylactic acid composite. Meanwhile, if the stirring speed exceeds 1000 rpm, excessive aggregation of the oil-in-water emulsion may be caused, resulting in a non-uniform particle size of the composite. The stereocomplex polylactic acid composite in the form of microparticles can be produced from the oil-in-water emulsion as a result of ring-opening polymerization between the poly(L-lactic acid) and the poly(D-lactic acid) at the water/oil phase interface. The stereocomplex polylactic acid composite is in the form of microspheres having an average particle diameter of 0.1 to 100 μm and has a melting point of 200 to 280° C. and a stereocomplexation efficiency of at least 95%.

Particularly, although not explicitly described in the Examples section that follows, stereocomplex polylactic acid composites were produced in the form of films by varying the components of the water phase solution and the oil phase solution, the mixing ratio between the water phase solution and the oil phase solution, the mixing time, the mixing speed, and the kind of the first nonionic surfactant and their elastic moduli, elongations, and tensile strengths were measured.

As a result, when the following conditions were all met, the stereocomplex polylactic acid composites were found to have improved crystallinities, resulting in increases in elastic modulus, elongation, and tensile strength, unlike when other conditions and other numerical ranges were employed. It was also found that the production times of the composites were considerably reduced and the production yields of the composites were improved.

(1) The water phase solution is mixed with the first nonionic surfactant in a weight ratio of 1:0.03-0.08, (2) the oil phase solution is prepared by mixing the poly(L-lactic acid) with the poly(D-lactic acid) in a weight ratio of 5:5 for 1 minute to 5 minutes in step (b), (3) the oil phase solution further includes the second nonionic surfactant, (4) the second nonionic surfactant is mixed in an amount of 1 to 6% by weight, based on the total weight of the oil phase solution, (5) each of the first nonionic surfactant and the second nonionic surfactant is polyoxyethylene sorbitan monolaurate, and (6) the oil-in-water (O/W) emulsion is prepared by adding dropwise the oil phase solution at a rate of 20 to 50 μl/s to the water phase solution with a micropipette.

When any one of the above six conditions was not met, unreacted polymers were melted and acted as impurities, the mechanical properties (including elastic moduli, elongations, and tensile strengths) of the stereocomplex polylactic acid composites deteriorated significantly, the production times of the stereocomplex polylactic acid composites increased, and the production yields of the stereocomplex polylactic acid composites dropped below 90%.

Although not explicitly described in the Examples section that follows, stereocomplex polylactic acid composites were produced that met the following conditions in addition to the above-mentioned conditions.

(7) The poly(L-lactic acid) has a number average molecular weight (Mn) of 100,000 to 120,000 g/mol and the poly(D-lactic acid) has a number average molecular weight (Mn) of 110,000 to 130,000 g/mol in step (b); (8) the oil-in-water emulsion is subjected to magnetic stirring at a speed of 600 to 900 rpm in step (d); and (9) the stereocomplex polylactic acid composite has an average particle diameter of 3.5 to 6 μm, a melting point of 215 to 240° C., and a stereocomplexation efficiency of at least 99%.

The stereocomplex polylactic acid composites did not lose their molecular weights even at high temperatures of 200° C. or more and their inherent characteristics were not impaired.

When any one of the above six conditions was not met, the polylactic acids were thermally degraded during polymerization at high temperature, resulting in a considerable reduction in molecular weight, and the composites lost their inherent characteristics, thus being unsuitable for use as implantable materials or components.

The present invention also provides a stereocomplex polylactic acid composite produced by mixing an oil phase solution including poly(L-lactic acid) and poly(D-lactic acid) with a water phase solution including water and a nonionic surfactant and subjecting the mixture to oil-in-water emulsion blending wherein the stereocomplex polylactic acid composite is in the form of microspheres having an average particle diameter of 0.1 to 100 μm and has a melting point of 200 to 280° C. and a stereocomplexation efficiency of at least 95%. The nonionic surfactant may be the same as the first nonionic surfactant.

The stereocomplex polylactic acid composite may have high thermal stability and improved physical and mechanical strength due to its high crystal melting point compared to the single-phase PLLA or PDLA. The stereocomplex polylactic acid composite has an average particle diameter of 0.1 to 100 μm, preferably 1 to 80 μm, more preferably 3 to 35 μm, most preferably 3.5 to 6 μm. The stereocomplex polylactic acid composite has a melting point of 200 to 280° C., preferably 210 to 260° C., more preferably 215 to 240° C., most preferably 230° C. The stereocomplex polylactic acid composite has a number average molecular weight of 50,000 to 400,000 g/mol, preferably 150,000 to 300,000 g/mol, most preferably 230,000 g/mol. The stereocomplex polylactic acid composite has a stereocomplexation efficiency of at least 95%, preferably at least 97%, most preferably at least 99%.

The poly(L-lactic acid) may have a number average molecular weight (Mn) of 50,000 to 150,000 g/mol and the poly(D-lactic acid) may have a number average molecular weight (Mn) of 50,000 to 150,000 g/mol. The stereocomplex polylactic acid composite may be a mixture of the poly(L-lactic acid) and the poly(D-lactic acid) in a weight ratio of 1:9 to 9:1.

The present invention also provides a method for preparing a drug delivery composition, including: (a) preparing a water phase solution including water and a first nonionic surfactant; (b) preparing an oil phase solution including poly(L-lactic acid), poly(D-lactic acid), and a drug; (c) dispersing the oil phase solution in the water phase solution while adding dropwise the oil phase solution at a rate of 1 to 200 μl/s to the water phase solution to prepare an oil-in-water (O/W) emulsion; and (d) stirring the oil-in-water emulsion to produce a stereocomplex polylactic acid composite loaded with the drug.

In step (b), the drug may be present in an amount of 0.1 to 33% by weight, based on the total weight of the oil phase solution and the mixing time may be 1 minute to 3 hours or 15 to 18 hours.

The drug is present in an amount of 0.1 to 33% by weight, preferably 1 to 33% by weight, 5 to 20% by weight, most preferably 5 to 10% by weight, based on the total weight of the oil phase solution. The presence of the drug in an amount exceeding 33% by weight may cause abnormal melting of the drug at around 75° C. and may inhibit the production of the stereocomplex polylactic acid composite.

The drug may be an anticancer agent. Specifically, the drug may be selected from the group consisting of fluorouracil (5-FU), capecitabine, cytarabine, gemcitabine, mercaptopurine, fludarabine, methotrexate, pemetrexed, and mixtures thereof. The drug is preferably selected from the group consisting of fluorouracil, capecitabine, mercaptopurine, and mixtures thereof. The drug is most preferably fluorouracil (5-FU). Fluorouracil (5-FU) is a drug that can be used to treat one or more cancers selected from the group consisting of breast cancer, colon cancer, rectal cancer, and pancreatic cancer. Fluorouracil (5-FU) has good thermal stability due to its high melting point (−282° C.).

In step (d), the oil-in-water emulsion is mixed using a magnetic stirrer at a high speed. This rapid mixing induces the production of the stereocomplex polylactic acid composite from the poly(L-lactic acid) and the poly(D-lactic acid) and enables the preparation of the drug delivery composition in which the stereocomplex polylactic acid composite is loaded with the drug. The drug can be infiltrated into the polymer chains of the stereocomplex polylactic acid during polymerization of the oil-in-water emulsion polymerization and can be loaded into the stereocomplex polylactic acid composite. The loaded drug can be released at room temperature over an extended period of 1 to 14 days.

The present invention also provides a drug delivery composition prepared by mixing an oil phase solution including poly(L-lactic acid), poly(D-lactic acid), and a drug with a water phase solution including water and a nonionic surfactant and subjecting the mixture to oil-in-water emulsion blending to produce a stereocomplex polylactic acid composite loaded with the drug wherein the stereocomplex polylactic acid composite is in the form of microspheres having an average particle diameter of 0.1 to 100 μm and has a melting point of 200 to 280° C. and a stereocomplexation efficiency of at least 95%.

The drug is present in an amount of 0.1 to 33% by weight, preferably 1 to 33% by weight, more preferably 5 to 20% by weight, most preferably 5 to 10% by weight, based on the total weight of the drug delivery composition.

The drug is loaded into the polymer chains of the stereocomplex polylactic acid and is easily released. Therefore, the drug delivery composition is applicable to a drug carrier.

As described above, the stereocomplex polylactic acid composite of the present invention is produced using oil-in-water emulsion blending that has the advantages of simple process, short production time, and high stereocomplexation efficiency compared to existing methods such as solution blending, melt-blending, and supercritical fluid technology.

In addition, the drug delivery composition using the stereocomplex polylactic acid composite according to the present invention is prepared using oil-in-water emulsion blending that allows the drug to be loaded into the polymer chains of the stereocomplex polylactic acid in a very easy and efficient manner simultaneously with the production of the stereocomplex polylactic acid, and facilitates release of the drug at room temperature over a long period of time. Furthermore, when one or more other substances are used in addition to the drug, secondary functions can be imparted to the stereocomplex polylactic acid-based polymer. Therefore, the present invention can greatly extend the application and utilization of polylactic acid, a representative environmentally friendly polymer.

The present invention will be more specifically explained with reference to the following examples but is not limited to these examples.

### Example 1: Production of Stereocomplex Polylactic Acid (Sc-PLA) Composites Using Oil-in-Water Emulsion Blending

[Materials]

Poly(L-lactic acid) (PLLA) having a number average molecular weight (Mn) of 110,000 g/mol to 120,000 g/mol and poly(D-lactic acid) (PDLA) having a number average molecular weight (Mn) of 110,000 g/mol to 120,000 g/mol were used to produce stereocomplex polylactic acid composites by a classical ring-opening polymerization method. Then, each of the PLLA and the PDLA was dissolved at a concentration of 1 g/25 mL in methylene chloride (CH₂Cl₂) before use. 5-FU as an anticancer drug was purchased from Sigma Aldrich.

Preparation of Emulsion 1

25 ml of Tween 20 (polyoxyethylene sorbitan monolaurate) having an HLB of 16-17 as a first nonionic surfactant was mixed with 500 ml of deionized water to prepare a water phase solution. Then, the PLLA solution and the PDLA solution were mixed in a weight ratio of 5:5 and the mixture was added dropwise at a rate of 100 μl/s to the water phase solution at 25° C. to prepare an oil-in-water emulsion. Subsequently, the oil-in-water emulsion was stirred at a speed of 800 rpm using a magnetic stirrer to produce a stereocomplex polylactic acid composite. Thereafter, the stereocomplex polylactic acid composite was washed twice with distilled water to remove foreign contaminants and dried in a vacuum oven to remove the solvent.

Preparation of Emulsion 2

A water phase solution was prepared as described in the preparation of Emulsion 1. Then, the PLLA solution and the PDLA solution in a weight ratio of 5:5 were placed in an empty beaker and homogenized by magnetic stirring for 24 h to prepare an oil phase solution where the production of a stereocomplex polylactic acid composite was induced. As described in the preparation of Emulsion 1, the oil phase solution was mixed with the water phase solution to produce a stereocomplex polylactic acid composite, which was then washed and dried.

Preparation of Emulsion 3

A water phase solution was prepared as described in the preparation of Emulsion 1. Then, the PLLA solution and the PDLA solution in a weight ratio of 5:5 were placed in an empty beaker and homogenized by magnetic stirring for 3 min to prepare an oil phase solution where the production of a stereocomplex polylactic acid composite was not induced. As described in the preparation of Emulsion 1, the oil phase solution was mixed with the water phase solution to produce a stereocomplex polylactic acid composite, which was then washed and dried.

Preparation of Emulsion 4

A water phase solution was prepared as described in the preparation of Emulsion 1. Then, the PLLA solution and the PDLA solution in a weight ratio of 5:5 were placed in an empty beaker. 97 ml of the mixed solution was added with 3 ml of Tween 20 as a second nonionic surfactant and homogenized by magnetic stirring for 5 min to prepare an oil phase solution. As described in the preparation of Emulsion 1, the oil phase solution was mixed with the water phase solution to produce a stereocomplex polylactic acid composite, which was then washed and dried.

Preparation of Emulsion 5

A water phase solution and an oil phase solution were prepared as described in the preparation of Emulsion 3. Then, the oil phase solution was mixed with the water phase solution while adding the oil phase solution drop by drop (50 μl/s) to the water phase solution with a micropipette, to produce a stereocomplex polylactic acid composite. The stereocomplex polylactic acid composite was washed and dried, as described in the preparation of Emulsion 1.

### Example 2: Preparation of Drug Delivery Composition Including Drug Loaded into Stereocomplex Polylactic Acid Composite (Sc-PLA/5-FU)

The anticancer drug 5-FU was dissolved at a concentration of 50 mg/25 ml in propylene glycol. A water phase solution and an oil phase solution were prepared as described in the preparation of Emulsion 5. 20% by weight of the drug was added to 100% by weight of the oil phase solution in an empty beaker and homogenized by magnetic stirring for 3 min to prepare an oil phase solution where the production of a stereocomplex polylactic acid composite was not induced. As described in the preparation of Emulsion 5, the oil phase solution was mixed with the water phase solution using a magnetic stirrer at a high speed while adding the oil phase solution drop by drop (50 μl/s) to the water phase solution with a micropipette, to prepare a drug delivery composition including a stereocomplex polylactic acid composite loaded with the anticancer agent (sc-PLA/5-FU).

### Comparative Example 1: Production of Stereocomplex Polylactic Acid Composite Using Solution Blending

A stereocomplex polylactic acid composite was produced using the same components as described in Example 1, except that solution blending was used instead of oil-in-water emulsion blending.

### Comparative Example 2: Production of Stereocomplex Polylactic Acid Composite Using Supercritical Fluid Technology (Conventional SCF)

A stereocomplex polylactic acid composite was produced using the same components as described in Example 1, except that conventional supercritical fluid (SCF) technology was used instead of oil-in-water emulsion blending.

### Comparative Example 3: Production of Stereocomplex Polylactic Acid

Composite Using Solution Feed Supercritical Fluid Technology (SCF) A stereocomplex polylactic acid composite was produced using the same components as described in Example 1, except that conventional supercritical fluid (SCF) technology was used instead of oil-in-water emulsion blending.

### Experimental Example 1: Thermal, Thermogravimetric, and Wide-Angle X-Ray Diffraction Analyses of the Stereocomplex Polylactic Acid (Sc-PLA) Composites

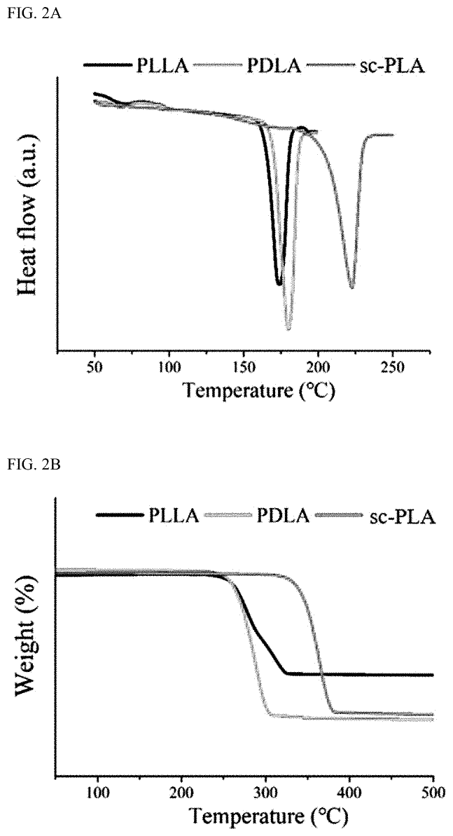

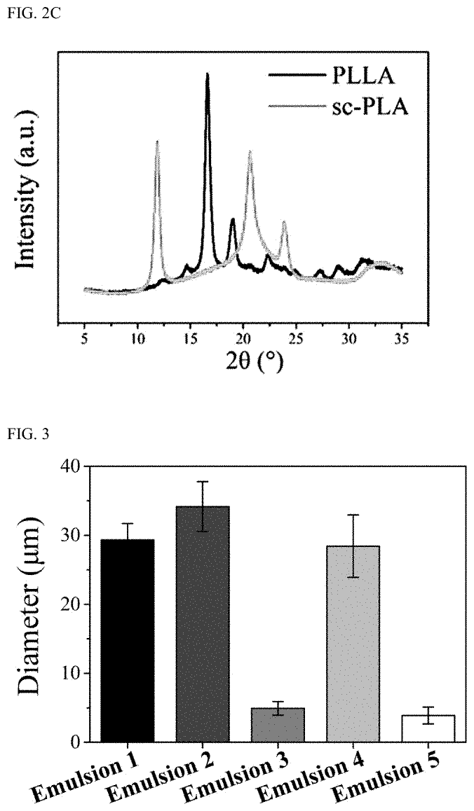

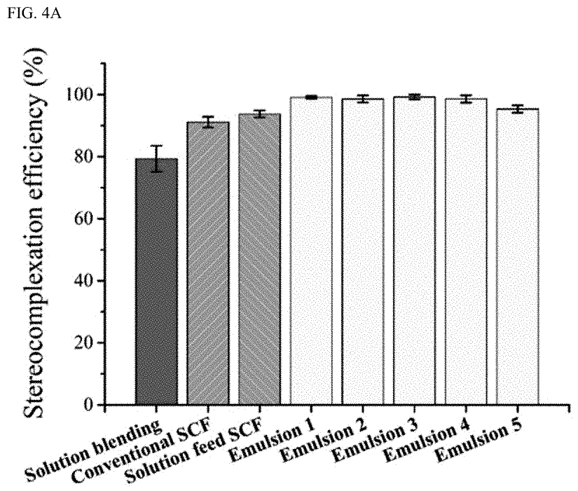

The thermal properties and crystal structures of the stereocomplex polylactic acid composites produced in Example 1 were analyzed by thermogravimetric analysis (TGA), differential scanning calorimetry (DSC), and wide-angle X-ray diffraction. 10 mg of the scPLA composite produced in Example 1 (Emulsion 5), 10 mg of the PLLA for comparison, and 10 mg of the PDLA for comparison were heated to 250° C. at a rate of 10° C./min and their thermal properties were measured by DSC. The results are shown in FIGS. 2A to 2C and 3.

FIG. 2A graphically shows the results of thermal analysis, FIG. 2B shows thermogravimetric analysis, and FIG. 2C shows wide-angle X-ray diffraction analysis for the scPLA composite produced in Example 1 (Emulsion 5), the PLLA, and the PDLA. As can be seen from FIG. 2A, the homopolymers PLLA and PDLA had melting points of 180° C. whereas the stereocomplex polylactic acid (sc-PLA) composite produced using oil-in-water emulsion blending in Example 1 (Emulsion 5) had a melting peak at around 230° C., which is higher by >50° C. than the melting points of the homopolymers. From FIG. 2B, it can also be seen that the sc-PLA composite had a higher thermal degradation onset temperature and better thermal stability than the PLLA and the PDLA. These results are typical thermal properties of stereocomplex polylactic acid composites, indicating successful production of the inventive stereocomplex polylactic acid composite via oil-in-water emulsion blending. The sc-PLA composite produced via oil-in-water emulsion blending was found to have a stereocomplex crystal structure different from the crystal structure of the PLLA (see FIG. 2C).

FIG. 3 shows the particle sizes of the sc-PLA composites produced in Example 1 (Emulsions 1-5). The sc-PLA composites produced in Example 1 (Emulsions 1-5) had particle sizes of 29.4 μm, 34.2 μm, 4.9 μm, 28.4 μm, and 3.9 μm, respectively, as measured by DSC. The sc-PLA composites of Emulsions 3 and 5 had smaller particle sizes than the other sc-PLA composites. The particles of the sc-PLA composite of Emulsion 5 were smallest and most uniform. The reason why the sc-PLA composite of Emulsion 5 had the smallest particle size was because the oil phase PLLA and PDLA solutions were added drop by drop to the water phase solution with a micropipette to make the polymer chains smaller, unlike in the other methods where the oil phase solutions were rapidly added to the water phase solution.

### Experimental Example 2: Analysis of Stereocomplexation Efficiencies and Synthesis Times of the Stereocomplex Polylactic Acid (Sc-PLA) Composites

The stereocomplexation efficiencies and synthesis times of the stereocomplex polylactic acid composites produced in Example 1 and Comparative Examples 1-3 were analyzed by TGA and DSC. Based on the DSC results, the stereocomplexation efficiency was calculated by the following equation:

Stereocomplexation efficiency (%)=ΔH_{m(sc-PLA)}/ΔH_{m(Homopolymers)},

where ΔH_{m(sc-PLA)} is the enthalpy of melting for the stereocomplex polylactic acid (sc-PLA) composite and ΔH_{m(Homopolymers)} represents the enthalpy of melting for the homopolymers PLLA and PDLA.

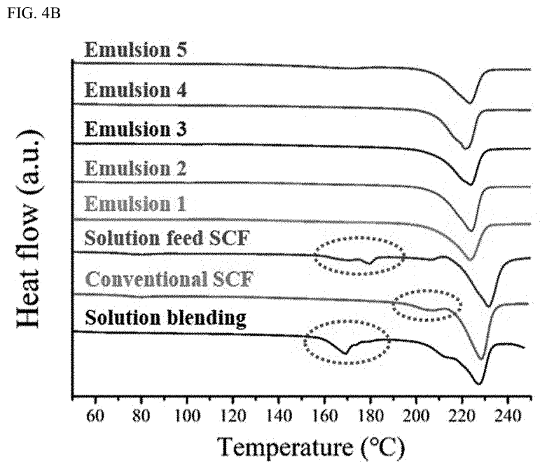

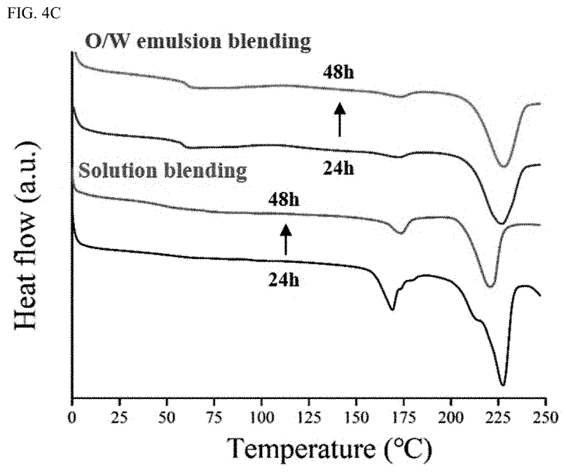

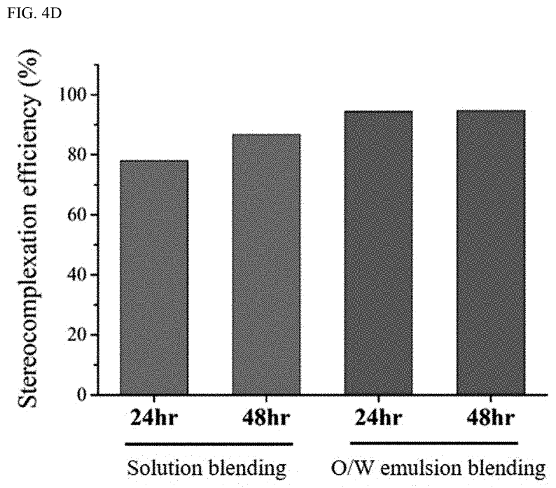

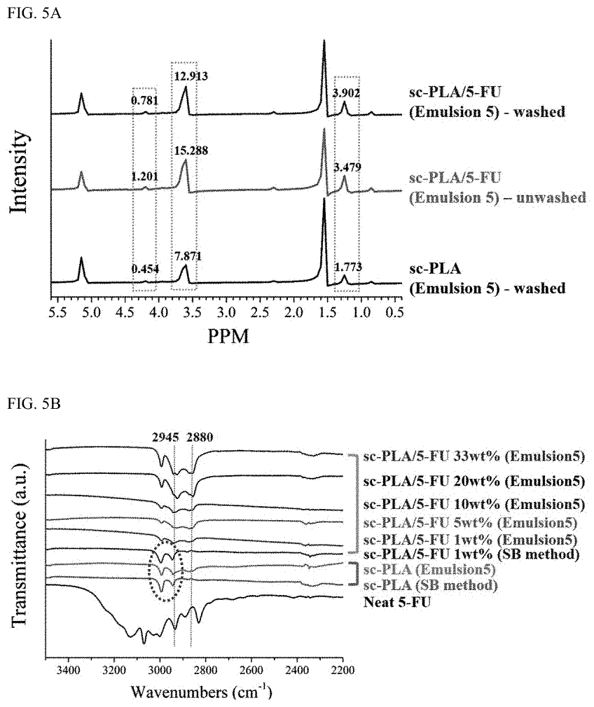

The results are shown in FIGS. 4A to 4D.

FIGS. 4A and 4D shows the stereocomplexation efficiencies of the stereocomplex polylactic acid composites produced in Example 1 and Comparative Examples 1-3 and FIGS. 4B and 4C shows the results of thermogravimetric analysis for the stereocomplex polylactic acid composites. As can be seen from FIG. 4A, the composite produced using solution blending (Comparative Example 1), which is the most widely method in the art, showed an efficiency of 79.3%, the composite produced using supercritical fluid technology (Comparative Example 2), which is usually used in the art, showed an efficiency of 91.1%, and the composite produced using improved solution feed supercritical fluid technology (Comparative Example 3) showed an efficiency of 93.7%. In contrast, the stereocomplex polylactic acid composites produced using oil-in-water emulsion blending (Example 1 (Emulsions 1-5)) showed a high efficiency close to a maximum of 99.2%.

As can be seen from FIG. 4B, polymers remaining unreacted were melted in the temperature ranges other than the melting points (230° C.) of the stereocomplex polylactic acid composites produced in Comparative Example 1 (solution blending), Comparative Example 2 (conventional SCF), and Comparative Example 3 (solution feed SCF). In contrast, no melting of foreign matter or unreacted polymers was observed in the temperature ranges other than the melding points (−230° C.) of the stereocomplex polylactic acid composites of Emulsions 1-5 (oil-in-water emulsion blending). From these results, it can be concluded that oil-in-water emulsion blending enables the production of stereocomplex polylactic acid composites in high yields compared to other methods.

FIGS. 4C and 4D compare the thermal properties and stereocomplexation efficiencies of the sc-PLA composites produced after synthesis for 24 h and 48 h in Comparative Example 1 (solution blending) and the sc-PLA composites produced after synthesis for 24 h and 48 h in Example 1 (Emulsion 5). Referring to FIGS. 4C and 4D, the production of the stereocomplex polylactic acid composites after synthesis for 24 h and 48 h in Example 1 (Emulsion 5) was induced with higher efficiency than the production of the sc-PLA composites after synthesis for 24 h and 48 h in Comparative Example 1, indicating that oil-in-water emulsion blending can be used to produce a stereocomplex polylactic acid composite with higher stereocomplexation efficiency in a much shorter time than other existing methods.

### Experimental Example 3: Determination of Whether the Anticancer Agent (5-FU) was Loaded into the Drug Delivery Composition (Sc-PLA/5-FU)

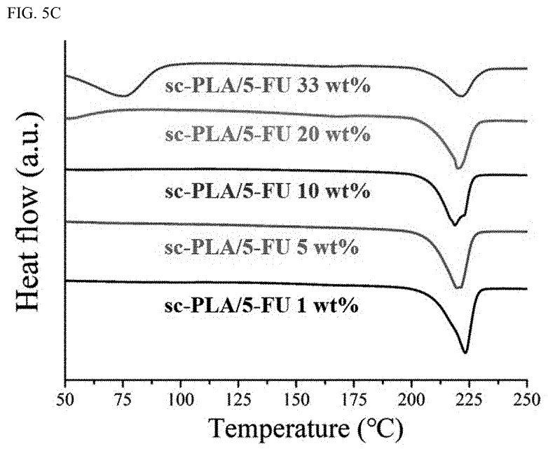

Proton nuclear magnetic resonance (¹H-NMR) spectra and Fourier transform infrared (FTIR) spectra were recorded to determine whether the secondary anticancer agent 5-FU was loaded into the drug delivery composition (sc-PLA/5-FU) prepared in Example 2. The results are shown in FIGS. 5A to 5C.

FIG. 5A shows ¹H-NMR spectra, FIG. 5B shows transmittances, and FIG. 5C shows DSC thermograms of the sc-PLA composite produced in Example 1 (Emulsion 5) and the drug delivery composition (sc-PLA/5-FU) prepared in Example 2. Referring to FIG. 5A, the anticancer-loaded drug delivery composition (washed, unwashed) had at least two-fold higher peak intensities at around 4.2 ppm, 3.6 ppm, and 1.25 ppm than the sc-PLA composite (Emulsion 5). The higher peak intensities are attributed to changes caused by the α-carbon, hydroxyl groups, and methyl groups in the molecules of the anticancer agent 5-FU and the peak intensities at the corresponding ppm values were still increased even after twice washing with distilled water.

In FIG. 5B, when the content of the anticancer agent 5-FU loaded into the drug delivery composition (sc-PLA/5-FU) increased from 1 wt % to 33 wt %, peaks observed at around 2995 cm⁻¹ and 2945 cm⁻¹ were shifted to lower wavenumbers with increased intensities. These peak shifts indicate asymmetric and symmetric CH₃ stretching and Cα-H stretching and were observed only in the stereocomplex polylactic acid composite produced using oil-in-water emulsion blending. In contrast, this phenomenon was not observed in the anticancer agent (5-FU)-loaded composition prepared using solution blending (SB method).

FIG. 5C confirms that the preparation of the sc-PLA/5-FU was successfully induced when the content of the anticancer agent 5-FU loaded into the drug delivery composition (sc-PLA/5-FU) was gradually increased to 1, 5, 10, 20, and 33 wt %. However, when the anticancer agent 5-FU was loaded in an amount exceeding 33 wt %, an abnormal melting peak was observed at around 75° C. In conclusion, the presence of the anticancer agent in an amount exceeding 33 wt % rather hinders the production of the stereocomplex polylactic acid composite.

### Experimental Example 4: Analysis of Release of the Drug from the Drug Delivery Composition (Sc-PLA/5-FU)

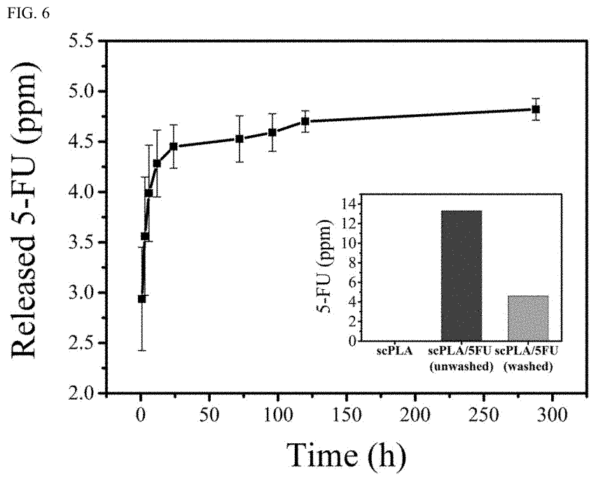

The time-dependent drug release profile of the drug delivery composition (sc-PLA/5-FU) prepared in Example 2 was analyzed by high-performance liquid chromatography (HPLC) to determine whether the composition indeed had an effect on the drug release. The results are shown in FIG. 6.

FIG. 6 shows the drug release profile of the anticancer agent (5-FU) from the drug delivery composition (sc-PLA/5-FU) prepared in Example 2, which was determined by HPLC. Referring first to the inset in FIG. 6, the drug was detected in the drug delivery composition (sc-PLA/5-FU) both before and after washing. In addition, the anticancer agent 5-FU loaded into the drug delivery composition (sc-PLA/5-FU) was normally released for 12 days after loading. These results concluded that oil-in-water emulsion blending enables the production of a stereocomplex polylactic acid composite with high efficiency and a secondary anticancer agent can be effectively loaded into and released from the composite, demonstrating the applicability of the composite to a drug carrier.

What is claimed is:

1. A method for producing a stereocomplex polylactic acid composite based on oil-in-water emulsion blending, comprising: (a) preparing a water phase solution comprising water and a first nonionic surfactant; (b) preparing an oil phase solution comprising poly(L-lactic acid) and poly(D-lactic acid); (c) dispersing the oil phase solution in the water phase solution while adding dropwise the oil phase solution at a rate of 1 to 200 μl/s to the water phase solution, to prepare an oil-in-water (O/W) emulsion; and (d) stirring the oil-in-water emulsion.

2. The method according to claim 1, wherein the water phase solution is prepared by mixing the water with the first nonionic surfactant in a weight ratio of 1:0.01-0.5.

3. The method according to claim 1, wherein, in step (b), the oil phase solution is prepared by mixing the poly(L-lactic acid) with the poly(D-lactic acid) in a weight ratio of 1:9 to 9:1 for 1 minute to 3 hours or for 15 hours to 28 hours.

4. The method according to claim 1, wherein the oil phase solution further comprises 0.1 to 10% by weight of a second nonionic surfactant, based on the total weight thereof.

5. The method according to claim 4, wherein the first nonionic surfactant and the second nonionic surfactant are each independently selected from the group consisting of polyoxyethylene sorbitan monolaurate, polyoxyethylene sorbitan monostearate, polyoxyethylene sorbitan monooleate, polyoxyethylene lauryl ether, polyoxyethylene cetyl ether, polyoxyethylene oleyl ether, polyoxyethylene, and mixtures thereof.

6. The method according to claim 1, wherein, in step (c), the oil-in-water (O/W) emulsion is prepared by adding dropwise the oil phase solution at a rate of 15 to 80 μl/s to the water phase solution with a micropipette.

7. The method according to claim 1, wherein, in step (d), the oil-in-water emulsion is subjected to magnetic stirring at a speed of 100 to 1000 rpm.

8. The method according to claim 1, wherein the stereocomplex polylactic acid composite is in the form of microspheres having an average particle diameter of 0.1 to 100 μm and has a melting point of 200 to 280° C. and a stereocomplexation efficiency of at least 95%.

9. The method according to claim 1, wherein the water phase solution is prepared by mixing the water with the first nonionic surfactant in a weight ratio of 1:0.03-0.08; the oil phase solution is prepared by mixing the poly(L-lactic acid) with the poly(D-lactic acid) in a weight ratio of 5:5 for 1 minute to 5 minutes in step (b); the oil phase solution further comprises 1 to 6% by weight of a second nonionic surfactant, based on the total weight thereof; each of the first nonionic surfactant and the second nonionic surfactant is polyoxyethylene sorbitan monolaurate; and the oil-in-water (O/W) emulsion is prepared by adding dropwise the oil phase solution at a rate of 20 to 50 μl/s to the water phase solution with a micropipette in step (c).
